(19) United States
(12) Patent Application Publication
(10) Pub. No.: US 2019/0265186 A1
(43) Pub. Date: Aug. 29, 2019

(54) ANALYTE SENSOR

(71) Applicant: PercuSense, Inc., VALENCIA, CA (US)

(72) Inventors: RAJIV SHAH, RANCHO PALOS VERDES, CA (US); BRADLEY C. LIANG, BLOOMFIELD HILLS, MI (US); ELLEN BOWMAN, PASADENA, CA (US); KATHERINE WOLFE, MISSISSAUGA (CA); Shuan Pendo, Wofford Heights, CA (US)

(73) Assignee: PercuSense, Inc., VALENCIA, CA (US)

(21) Appl. No.: 16/152,727

(22) Filed: Oct. 5, 2018

Related U.S. Application Data
(60) Provisional application No. 62/635,897, filed on Feb. 27, 2018.
(60) Provisional application No. 62/666,219, filed on May 3, 2018.

(51) Int. Cl. G01N 27/327; C12Q 1/00

(52) U.S. Cl. CPC G01N 27/3277; G01N 27/3272; C12Q 1/001; G01N 27/3276

(57) ABSTRACT

An electrode measuring the presence of an analyte is disclosed. The electrode includes a working conductor with an electrode reactive surface. The working electrode further includes a first reactive chemistry that is responsive to a first analyte. Additionally, the working electrode includes a first transport material that enables flux of the first analyte to the first reactive chemistry. Further included with the electrodes is a separation chemistry between the first reactive chemistry and the first transport material, the separation chemistry minimizing mixing of the first reactive chemistry and the first transport material.

### RELATED APPLICATIONS

This application claims the benefit of U.S. provisional application numbers: 62/635,897 filed Feb. 27, 2018; and 62/666,219 filed May 3, 2018. The applications listed above are hereby incorporated by reference in their entireties for all purposes.

### FIELD OF THE INVENTION

The present invention is generally directed to devices and methods that perform in vivo monitoring of an analyte. In particular, the devices and methods are for an electrochemical sensor that provides information regarding the amount of analyte within interstitial fluid of a subject.

### BACKGROUND OF THE INVENTION

Monitoring of particular analytes within a subject can be critically important to short-term and long-term well being. For example, the monitoring of glucose can be particularly important for people with diabetes in order to determine insulin or glucose requirements. In another example, the monitoring of lactate in postoperative patients can provide critical information regarding the detection and treatment of sepsis.

The need to perform continuous or near continuous analyte monitoring has resulted in the development of a variety of devices and methods. Some methods place electrochemical sensor devices designed to detect the desired analyte in blood vessels while other methods place the devices in subcutaneous or interstitial fluid. Both placement locations can provide challenges to receiving consistently valid data. Furthermore, achieving consistent placement location can be critical to hydrating, conditioning and calibrating the device before actual use. Hydrating and conditioning of commercially available sensor devices can be a time consuming process often taking fractions of hours up to multiple hours. Assuming the hydrating and conditioning process is completed successfully, a subject may have to compromise their freedom of movement or range of movement in order to keep the sensor properly located within their body.

Many advances have been made resulting in commercially available real-time glucose sensors. However, commercially available glucose sensors are unfortunately limited to determining concentrations of only glucose. Monitoring additional analytes within interstitial fluid can provide greater insight thereby enabling improved therapy resulting in improved outcomes. One difficulty encountered when electrochemically monitoring analyte levels within a subject is availability of stable reactants to enable reliable detection and monitoring of the analyte. Commercially available glucose sensors rely on oxidase based reactants such as glucose oxidase. Presently, oxidase reactants are not available for measuring every analyte of interest. In these situations, it may be necessary to use dehydrogenase based reactant. Because the endogenous concentrations of cofactors for dehydrogenase based reactants are relatively low, especially in comparison to endogenous cofactors for oxidase based reactants, commonly implemented structures for commercially available oxidase based sensors may have difficulty being adapted to function with dehydrogenase based reactants.

The claimed invention seeks to address many of the issues discussed above regarding in vivo monitoring of analytes using dehydrogenase based reactants. In many examples discussed below the analyte being measured is a ketone identified as 3-hydroxybutyrate (3HB). However, while specific embodiments and examples may be discussed regarding 3HB, the scope of the disclosure and claims should not be construed to be limited to 3HB. Rather it should be recognized that chemistry applied to sensors described herein is determinative of the analyte the sensor measures.

### BRIEF SUMMARY OF THE INVENTION

An electrode measuring the presence of an analyte is described as one embodiment. The electrode includes a working conductor with an electrode reactive surface. The working electrode further includes a first reactive chemistry that is responsive to a first analyte. Additionally, the working electrode includes a first transport material that enables flux of the first analyte to the first reactive chemistry. Further included with the electrodes is a separation chemistry between the first reactive chemistry and the first transport material, the separation chemistry minimizing mixing of the first reactive chemistry and the first transport material.

In another embodiment, a method to manufacture an electrode is described. The method to manufacture an electrode includes operations that pattern a conductor material to generate a working conductor. The method to manufacture an electrodes further includes an operation that creates a reactive surface on the working conductor. Operations that apply an interference reduction material over the reactive surface and apply a first reactive chemistry over the interference reduction material are also included within the method to manufacture an electrode. Additionally, the method includes operations that apply a first transport material over the first reactive chemistry and apply a second transport material over the first transport material.

Other features and advantages of the invention will become apparent from the following detailed description, taken in conjunction with the accompanying drawings that illustrate, by way of example, various features of embodiments of the invention.

### BRIEF DESCRIPTION OF THE DRAWINGS

FIG. 1A is a top view of an exemplary sensor assembly having multiple transducers.

FIGS. 1B and 1C are exemplary cross-sections illustrations of the multilayer structure of the transducer within the sensor assembly.

FIG. 2A is an exemplary illustration of analytes entering the sensor assembly via the first transport material when the sensor assembly is exposed to fluid within a subject.

FIG. 2B is an exemplary illustration of the generation of product analyte and migration of the product analyte to the working conductor.

FIG. 2C is an exemplary illustration intended to visually depict liberation of two electrons by the electrochemical oxidation of NADH on the working conductor, in accordance with embodiments of the present invention.

FIG. 3A is an exemplary illustration of an embodiment utilizing amplifying electrodes as a cofactor enhancing feature.

FIGS. 3B-1 through 3B-9 are exemplary illustrations of embodiments that include a second reactive chemistry as a cofactor enhancing feature, in accordance with embodiments of the present invention.

FIGS. 4A, 4B-1 and 4B-2 are alternative embodiments where the transducer structure is based on ring transducers, in accordance with embodiments of the present invention.

FIG. 5 is an exemplary flow chart illustrating operation to create a sensor assembly similar to what is illustrated in FIG. 3B-5, in accordance with embodiments of the present invention.

FIGS. 6A-6E are exemplary illustration of multianalyte sensor assemblies, in accordance with embodiments of the present invention.

### DETAILED DESCRIPTION

Dehydrogenase based reactants for analytes of interest often require a cofactor such as, but not limited to nicotinamide adenine dinucleotide (NAD) or flavin adenine dinucleotide (FAD). Because these cofactors are found in very limited concentrations endogenously, it can be difficult to enable linearity and sensitivity of a transducer across a dynamic biologically relevant range. Described below are embodiments of a dehydrogenase based transducer that enables linear sensor response across a relevant dynamic range. In some embodiments endogenous cofactor is supplemented by doping or entrapping cofactor within the transducer structure. In other embodiments, cofactor is generated from an endogenous analyte other than the analyte being measured. The embodiments described below are intended to be exemplary rather than limiting. Furthermore, the principles of operation of the various embodiments should be viewed as interchangeable or combinable with other embodiments insofar as the structure being modified remains functional for its intended purpose.

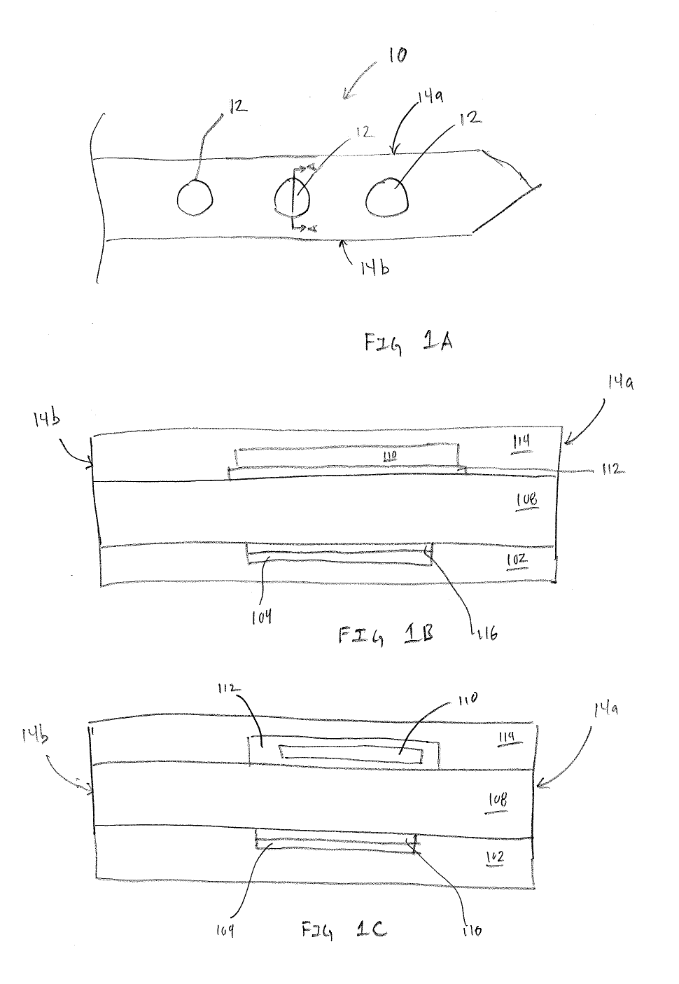

FIG. 1A is a top view of an exemplary sensor assembly 10 having multiple transducers 12, in accordance with embodiments of the present invention. The sensor assembly 10 has a proximal end 10a, distal end 10b, along with edges 14a and 14b. As this disclosure is primarily directed toward the transducer 12, the proximal end 10a is illustrated without the typical contact pads that enable the sensor assembly 10 to be connected to an electronics package that enables operation and data acquisition, storage and transmission of data acquired by the sensor assembly 10. The distal end 10b is illustrated as a symmetrical needle point or spear point in order to have the sensor assembly 10 assist during the insertion process. However, in other embodiments the distal end 10b can take alternative shapes, such as, but not limited to chisel tips, compound bevels, and a variety of asymmetrical tips that are configured to assist in piercing and cutting during insertion of the sensor assembly 10 within a percutaneous space.

Included within the sensor assembly 10 are a plurality of transducers 12 that are formed via a multilayer structure. The specific number of transducers 12 shown in FIG. 1A is intended to be exemplary rather than restrictive. In various embodiments fewer or additional transducers 12 are formed on the sensor assembly 10. Additionally, the transducers 12 shown in FIG. 1A are configured to measure a single analyte or metric, such as, but not limited to glucose, lactate, reactive oxygen species (ROS) ketones, or oxygen. In many embodiments a single sensor assembly 10 includes multiple sets of transducers, each set of transducers configured to measure a different analyte, metric, or electrochemically active molecule. For example, on a single sensor assembly 10, there may be sets of transducers configured to measure glucose, lactate and ketone. In other embodiments, the types and number of transducers configured to measure different analytes or metrics is only constrained by the size of the sensor assembly 10, the size of the transducer 12, and the size of the electrical traces required for each working conductor.

FIGS. 1B and 1C are exemplary cross-sections illustrations of the multilayer structure of the transducer 12 within the sensor assembly 10, in accordance with embodiments of the present invention. Each embodiments includes a working electrode 104 within insulation 102. The working conductor 104 can be formed using materials such as, but not limited to stainless steel or other electrically conductive materials. One benefit of forming the working conductor 104 from stainless steel is the ability to select a stainless steel with desirable mechanical properties such as toughness and elastic modulus.

Additionally, the working conductor 104 includes a reactive surface 116. In some embodiments, where the working conductor 104 is an electrically conductive material, the reactive surface 116 may simply an exposed surface of the working conductor 104. In other embodiments the reactive surface 116 is optionally formed on the working conductor 104 via a process or combination of processes such as, but not limited to, electroplating, printing, vapor deposition or the like. Specific embodiments of the reactive surface 116 include single or multiple layers of at least one or more materials such as, but not limited to graphene, graphene oxide, platinum, silver, gold, or other materials having desirable electrochemical properties. In some embodiments the reactive surface is formed on the working conductor 104 via a printing process such as, but not limited to screen printing or inkjet printing. In other embodiments, an electrodeposition process is used to create the reactive surface 116 on the working conductor 104. In one embodiment of a multilayer reactive surface 116, the reactive surface 116 includes a platinized surface over graphene, or a graphene oxide, iridium oxide, or iridium-carbon surface that is applied over a platinum layer. This structure is capable of operating at lower electrical potentials in order to exclude effects of interfering electroactive compounds.

An additional element to the transducer 12 is a first transport material 108. In many embodiments the first transport material 108 is selected from group of materials such as, but not limited to hydrogels. The first transport material 108 is intended to enable transport of analyte within fluid surrounding the sensor assembly 10 (FIG. 1) to the transducer 12. The first transport material 108 extends from edge 14a, across the sensor assembly 10 to edge 14b and enables analyte to be laterally transported from edges 14a and 14b toward, and across the working conductor 104. Transportation of analytes laterally from edges 14a and 14b creates a relatively long diffusion pathway. In many embodiments the long diffusion pathway enables analytes within the first transport material 108 to interact with optional chemistries or other structures within the transducer 12. In many embodiments, the first transport material 108 is selected from a family of biocompatible hydrogels. In some embodiments, the first transport material 108 may be a single hydrogel or a combination of multiple hydrogels. In the various embodiments, each hydrogel or combinations of hydrogels can be selected based on various physical or chemical properties such as, but not limited to swelling, cure time, hydration time, adhesion, durability, flexibility and the like.

The transducer 12 further includes a second transport material 114. In many embodiments, the second transport material 114 is selected from hydrophobic materials such as, but not limited to silicone. One benefit of using hydrophobic materials for the second transport material 114 is the ability to create a no flux boundary between the first transport material 108 and the second transport material 114. Confining analyte flux within the first transport material 108 can help define the lateral movement of analyte from the edges 14a and 14b toward the working electrode 104. An additional benefit of using hydrophobic materials such as silicone for the second transport material 114 is the ability . . . .

Between the first transport material 108 and the second transport material 114, or between the working conductor reactive surface 116 and the first transport layer 108, and being positioned at least over the working conductor 104, is a first reactive chemistry 110 and a separation chemistry 112. The first reactive chemistry 110, in many embodiments, is a mixture of reagent to interact with the desired analyte and a hydrogel. For example, if the analyte to be measured is glucose, one embodiments of the first reactive chemistry 110 would be a mixture of glucose oxidase and a hydrogel. In still other embodiments, the first reactive chemistry 110 is a combination of reagent, cofactor, and hydrogel. The inclusion of an optional cofactor within the first reactive chemistry enables detection and measurements of analytes using reagents such as, but not limited to those within the dehydrogenase family. An additional benefit of incorporating the cofactor into the first reactive chemistry is improving response time and linearity of the sensor across an operational range. For example, if the analyte being measured is 3-hydroxybutyrate (3HB), the reactive chemistry may include a reagent such as 3-hydroxybutyrate dehydrogenase (3HBDH) and a cofactor such be Nicotinamide Adenine Dinucleotide (NAD⁺), both being mixed with a hydrogel.

Mixing the reagent with a hydrogel enables even dispersion of the reagent and optional cofactor when it is applied within the transducer 12. In many embodiments, the hydrogel component within the first reactive chemistry can be cured with full, or maximum, crosslinking when it is exposed to specific wavelengths of light. Alternatively, if not exposed to the specific wavelength of light, the hydrogel component can be dried without maximum crosslinking by exposing the uncured hydrogel to heat, or simply letting water content of the hydrogel evaporate. In some embodiments, the first reactive chemistry 110 is not fully crosslinked. By not fully crosslinking the hydrogel, the reagent and optional cofactor can more easily move or migrate within the first reactive chemistry upon rehydration within a subject.

The purpose of the separation chemistry 112 is to minimize potential mixing of the first reactive chemistry 110 and the first transport material 108. Accordingly, in FIG. 1B the separation chemistry 112 is applied directly between the first transport materials 108 and the first reactive chemistry 110. However, in FIG. 1C, the separation chemistry 112 encapsulates the first reactive chemistry 110 further minimizing potential mixing of the first reactive chemistry 110 with both the first transport material 108 and the second transport material 114. The separation chemistry 112 is not intended to prevent movement of analyte or other molecules between the first transport material 108 and the first reactive chemistry 110. Rather, the separation chemistry 112 is intended to prevent, or minimize intermingling, or mixing of the first transport material 108 and the first reactive chemistry 110. To accomplish this goal, in many embodiments, the separation chemistry 112 is applied on top of the first reactive chemistry 108 and fully crosslinked/cured. The fully crosslinked/cured separation chemistry 112 can be selected based on characteristics such as, but not limited analyte transmissibility when fully crosslinked, cure time, swelling and the like.

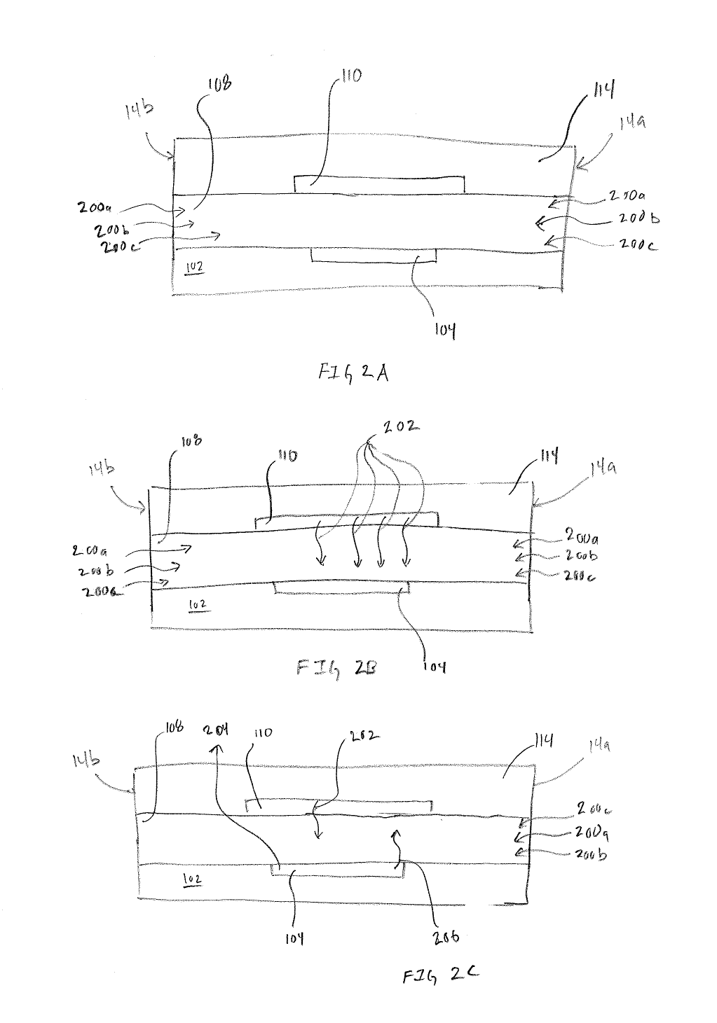

FIGS. 2A-2C are exemplary illustration of analyte movement, reactions and reaction product movement toward and within a transducer 12 with a dehydrogenase based reactive chemistry, in accordance with an embodiment of the present invention. Further description and discussion of FIGS. 2A-2C will be focused on the transducer 12 being configured to measure 3HB based on the following reaction in the presence of 3HBDH and cofactor NAD⁺.

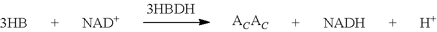

The discussion regarding 3HB detection and measurement is intended to be exemplary. Other embodiments of the sensor assembly and transducer can be configured to measure analytes other the 3HB using electrochemical enzymes from at least the oxidase or dehydrogenase family. Still other embodiments, modifications to the transducer 12 can enable detection of analytes using electrochemical enzymes from other families, such as, but not limited to X. Additionally, for simplicity, FIGS. 2A-2C do not contain illustrations for the reactive surface 116 and the separation chemistry 112 described in FIGS. 1B & 1C.

FIG. 2A is an exemplary illustration of analytes 200a, 200b and 200c entering the sensor assembly 10 via the first transport material 108 exposed to fluid within a subject along sides 14a and 14b, in accordance with embodiments of the present invention. Analyte 200a can be considered 3HB. Similarly, analyte 200b can be considered cofactor NAD⁺ while analyte 200c can be viewed as NADH. Each analyte 200a, 200b, and 200c laterally traverses from the edges 14a and 14b toward the first reactive chemistry 110. As previously discussed, the first reactive chemistry 110 includes 3HBDH suspended in a hydrogel. In other embodiments, the first reactive chemistry 110 includes 3HBDH along with an optional cofactor, in this case, NAD⁺. The inclusion of the cofactor within the first reactive chemistry 110 can be to overcome endogenous deficiencies. Specific to detecting 3HB, endogenous production of NAD⁺ is significantly less than 3HB. Accordingly, doping the first reactive chemistry 110 with the optional cofactor ensures sufficient NAD⁺ to completely react with the 3HB that is being measured. Regardless of whether the first reactive chemistry 110 includes the optional cofactor, the first reactive chemistry can be cured to either a fully cross linked condition or a partially cross linked condition. Partially cross linking the first reactive chemistry 110 may provide some benefit because reagent and optional cofactor may be able to more freely migrate within the first reactive chemistry 110.

FIG. 2B is an exemplary illustration of the generation of product analyte 202 and migration of the product analyte 202 to the working conductor 104, in accordance with embodiments of the present invention. As described above, there are multiple products of the 3HB and NAD⁺ reaction in the presence of 3HBDH. However, the product analyte 202 of interest is NADH. NADH is of interest, because as illustrated in FIG. 2C, NADH can be oxidized on the working conductor according to the following chemical reaction:

NADH→NAD⁺+H⁺2e⁻

FIG. 2C is an exemplary illustration intended to visually depict liberation of two electrons by the electrochemical oxidation of NADH on the working conductor 104, in accordance with embodiments of the present invention. The liberated electrons are illustrated as reaction product 204. In three electrode embodiments, the reaction product 204 is attracted to a reference electrode. In two electrode embodiments, the reaction product 204 is attracted to a pseudo-reference electrode. In both cases, the reaction product 204 roughly corresponds to the amount of 3HB within the fluid surrounding the sensor assembly. Recall that analyte 200c is endogenous NADH which can generate background signal not associated with the detection of 3HB. Eliminating the background signal from endogenous NADH can enable a more accurate correlation of signal generated by 3HB.

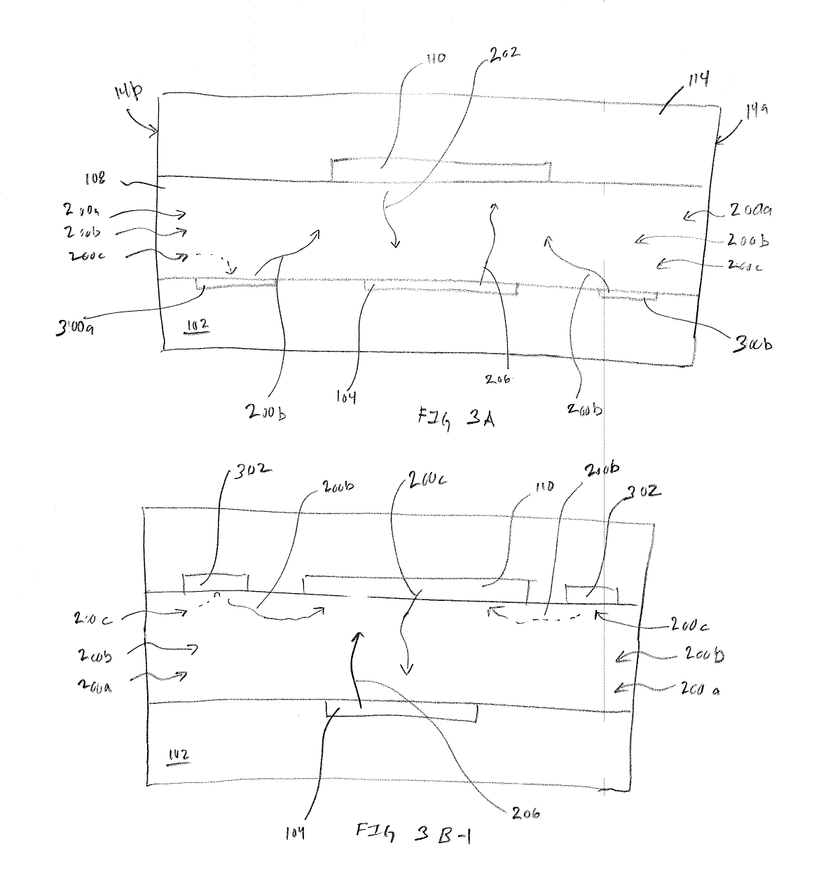

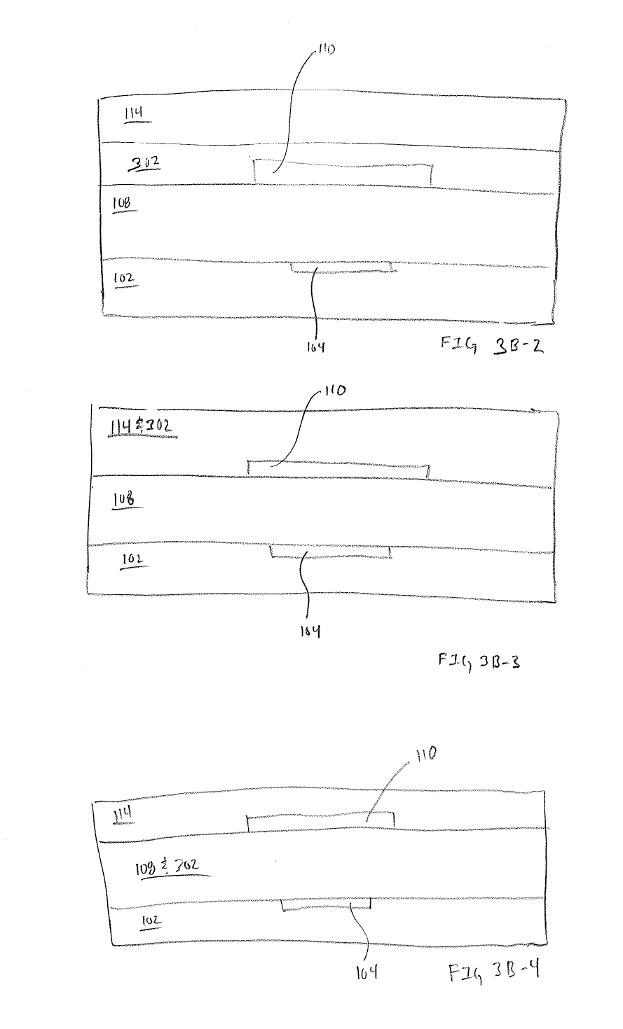

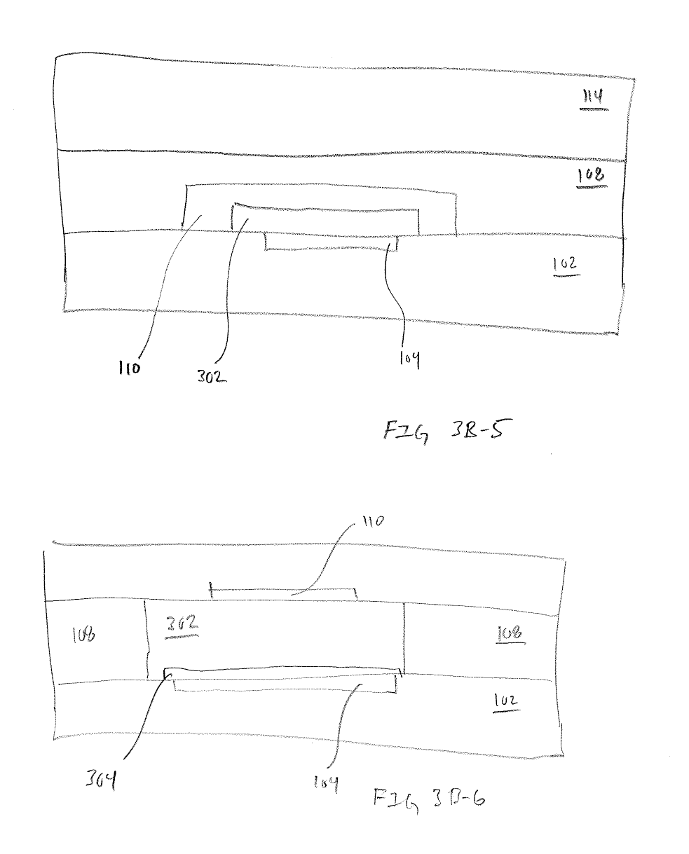

FIG. 3A is an exemplary illustration of an embodiment utilizing electrodes 300a and 300b as a cofactor enhancing feature, in accordance with embodiments of the present invention. The electrodes 300a and 300b are intended to oxidize an analyte such as endogenous NADH to generate NAD⁺ that can be used in the reaction between 3HB and 3HBDH within the first reactive chemistry 110. For clarity, the reactive surface 116 is not illustrated on either the working conductor 104 or the electrodes 300a/300b. Additionally, the separation chemistry 112 is also not illustrated. However, it should be understood that elements or features described in other figures can be combined or included with subsequent or prior embodiments.

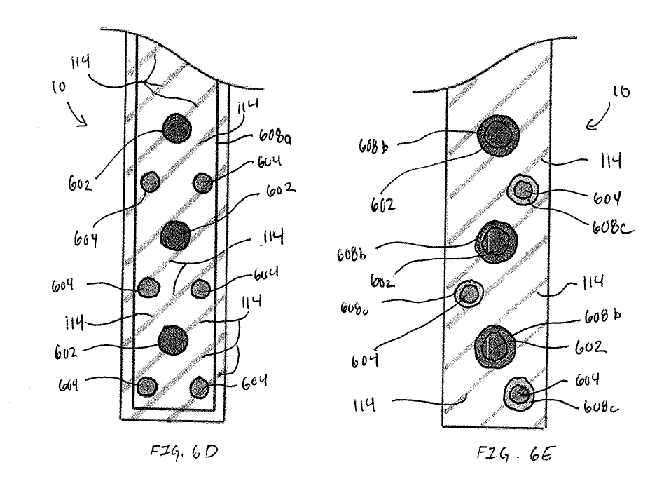

FIGS. 3B-1 through 3B-9 are exemplary illustrations of embodiments that include a second reactive chemistry 302 as a cofactor enhancing feature, in accordance with embodiments of the pre sent invention. In FIG. 3B-1 a second reactive chemistry 302 is located closer to edges 14a and 14b and is intended to react with at least one endogenous analyte in order to both eliminate background noise and create additional cofactor for consumption via the first reactive chemistry 110. In embodiments intended to measure ketones, the second reactive chemistry 302 can be a mixture of NADH-oxidase and a biocompatible hydrogel. In operation, the second reactive chemistry, in many embodiments, NADH-oxidase will react with endogenous NADH to create NAD⁺ that can be consumed when the measured analyte 3HB reacts with the first reactive chemistry 110 or 3HBDH.

A potential side effect of using the second reactive chemistry 302 is the generation of interfering compounds. For example, in many embodiments, the second reactive chemistry 302 may generate peroxide or other electroactive species which may be oxidized by the working conductor 104. In some embodiments, compensation for interfering compounds, either endogenous or generated via a reaction within the sensor assembly, is achieved using interference reduction material 304. Exemplary, non-restrictive examples of interference reduction material (IRM) 304 include, but are not limited to chemistries and curable materials. Catalase is an example of a chemistry that can be used as an IRM 304 because the catalase enzyme catalyzes the decomposition of hydrogen peroxide (generated via reaction between endogenous analytes and the second reactive chemistry). Other examples of chemistry based IRM 304 includes chemistries designed or configured to consume undesirable compounds, such as, but not limited to acetaminophen. Curable materials such as hydrogels can also be used as an IRM 304 by selecting or tuning the hydrogel to crosslink with preferred porosity that enables or restricts transport molecules of a particular size. Positively- or negatively-doped materials can be used to enable or restrict transport of charged molecules of a particular charge. Though discussed separately, some embodiments of the IRM are configured or tuned to compensate for single or multiple interfering compounds using combinations of a single chemistry or multiple chemistries and/or a single curable material or multiple curable materials.

In some embodiments, especially single analyte sensor configurations, the IRM 304 can be mixed with the first transport material 108, as shown in FIG. 3B-1. In still other embodiments, IRM 304 may be selectively placed in close proximity, completely encapsulate, or even substantially encapsulate the second reactive chemistry 302, as illustrated in FIG. 3B-7. The rationale for placing IRM 304 in close proximity to the second reactive chemistry 302 being IRM 304 is needed most where a reaction with the second reactive chemistry is producing an interfering compound. In still other embodiments, the IRM 304 is selectively placed over the working conductor 104 to prevent interfering compounds from interacting with the oxidation reaction, as shown in FIG. 3B-6. While some embodiments illustrated in FIGS. 3B-2 through 3B-9 have explicitly located IRM 304, it should be understood that IRM 304 can be extensively used across the entire sensor assembly or in specific locations that either target production of an interfering compound or protect the working conductor from a specific interfering compound. Accordingly, the embodiments illustrated in FIGS. 3B-2 through 3B-9 can each optionally incorporate an IRM 304 within the sensor assembly. In embodiments where the sensor assembly is configured to measure multiple analytes, selective placement of IRM 304 may be necessary to avoid impacting signal from other analytes.

FIG. 3B-2 is an additional embodiment of using a second reactive chemistry 302 as a cofactor enhancing feature, in accordance with embodiments of the present invention. In FIG. 3B-2, the second reactive chemistry 302 is applied in a discrete layer over the first reactive chemistry 110 while being between the first transport material 108 and the second transport material 114. Because the second reactive chemistry 302 may include a reagent such as NADH-oxidase distributed throughout a hydrogel, endogenous analyte such as NADH will be consumed before it can be oxidized via the working electrode 104. Additionally, the NAD⁺ resulting from the NADH/NADH-oxidase reaction can be further utilized in the reaction between 3HB, the analyte being measured, and the first reactive chemistry 110.

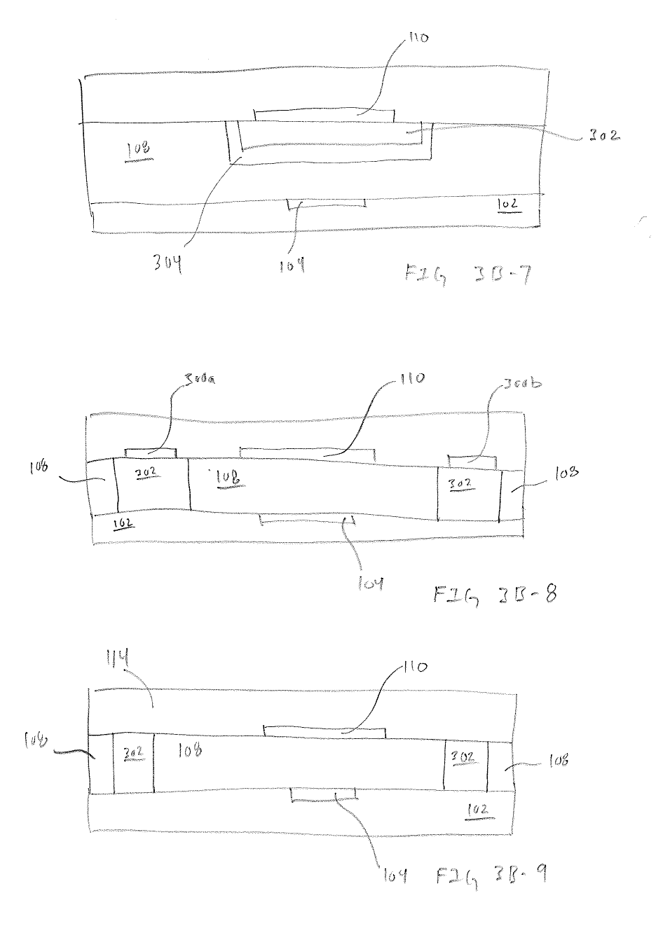

FIGS. 3B-3 and 3B-4 are additional exemplary cofactor enhancing feature embodiments having a second reactive chemistry 302 that may or may not include an interference reduction material 304, in accordance with embodiments of the present invention. In FIG. 3B-3, the second reactive chemistry 302 is mixed with the second transport material 114. In FIG. 3B-4, the second reactive chemistry 302 is mixed with the first transport material 108. The second transport material 114 can be selected from hydrophobic materials such as, but not limited to silicone. One benefit of using hydrophobic materials for the second transport material 114 is the ability to create a no flux boundary between the first transport material 108 and the second transport material 114. The no flux boundary confines or restricts fluid flow within the first transport material 108 resulting in analyte such as endogenous NADH reacting with the second reactive chemistry within the first transport material 108. In alternative embodiments the second transport material 114 is selected from hydrophilic materials such as, but not limited to hydrogels.

FIG. 3B-5 is an alternative embodiment where both the first reactive chemistry 110 and optionally the second reactive chemistry 302 are moved closer to the working electrode 104, in accordance with embodiments of the present invention. FIGS. 3B-6-3B-9 are additional exemplary embodiments that are not intended to be limiting, for example, FIG. 3B-6 includes selective placement of the second reactive chemistry 302 and an interference reduction material 304 between the first reactive chemistry 110 and the working conductor 104. Placement of the IRM 304 over the working conductor 104 decreases interference from interfering compound generated by the second reactive chemistry and endogenous analytes. In FIG. 3B-7, the IRM 304 partially encapsulates the second reactive chemistry 302 preventing interfering compounds created by a reaction with the second reactive chemistry 302 from reaching the working conductor 104.

FIG. 3B-8 is an exemplary embodiment that combines electrical and chemical cofactor enhancing features, in accordance with embodiments of the present invention. FIG. 3B-8 includes electrodes 300a and 300b in addition to second reactive chemistry 302. In some embodiments the electrodes 300a and 300b are configured to oxidize endogenous analyte. However, in other embodiments, the electrodes 300a and 300b are configured to oxidize byproduct of a reaction between endogenous analyte and the second reactive chemistry. For example, in some embodiments the electrodes 300a and 300b oxidize hydrogen peroxide, a byproduct of an interfering endogenous analyte and the second reactive chemistry 302.

FIG. 3B-9 is an exemplary embodiments utilizing selectively applied second reactive chemistry 302 as a cofactor enhancing feature. FIG. 3B-9 represents an embodiment that easily demonstrates how lateral diffusion of analytes from the edges 14a/14b toward the reactive chemistry 110 and working conductor 104 provides a pathway length to manipulate interfering analytes. Specifically, compared to sensor designs where diffusion of analytes is normal to the reactive surface of a working conductor, the lateral diffusion illustrated in FIGS. 3A through 3B-9 provides significantly greater path lengths that enable either chemical or electrochemical interaction with interfering analytes prior to their exposure to either the first reactive chemistry 110 or the working conductor 104.

FIGS. 4A and 4B-1 are alternative embodiments where the transducer structure is based on aperture transducers, in accordance with embodiments of the present invention. Additional disclosure regarding aperture, or ring, transducers can be found in U.S. patent application Ser. No. 15/472,194, filed on Mar. 28, 2017, that is herein incorporated by reference for all purposes. While the physical structure of the aperture electrode may differ from those described above, the principles of operation remain similar. Specifically, reacting an analyte of interest with a first reactive chemistry from the dehydrogenase family, with or without an optional cofactor to generate an analyte that is oxidized via the working conductor.

FIG. 4B-2 is an exemplary illustration of another embodiment of an aperture electrode configured to accommodate different chemistries, or combinations of chemistries at the location identified as Ω 402. For example, in various embodiments Ω 402 can be chemistries such as, but it not limited to, second transport material 108, second reactive chemistry 302, IRM 304, a combination of second reactive chemistry and IRM, and a combination of second transport material 108 and second reactive chemistry. In various other embodiments Ω 402 is another material or combination of materials such as, but not limited to first transport material 104, second reactive chemistry 302, IRM 304 or other materials described herein. Additionally, while the embodiments illustrated in FIG. 4B-2 has particular chemistries in various locations, the various locations should not be construed as limiting. Various chemistries or combinations of chemistries can be placed in different locations or within different layers of the aperture or any other electrode structure described herein to tune or optimize performance of the transducer.

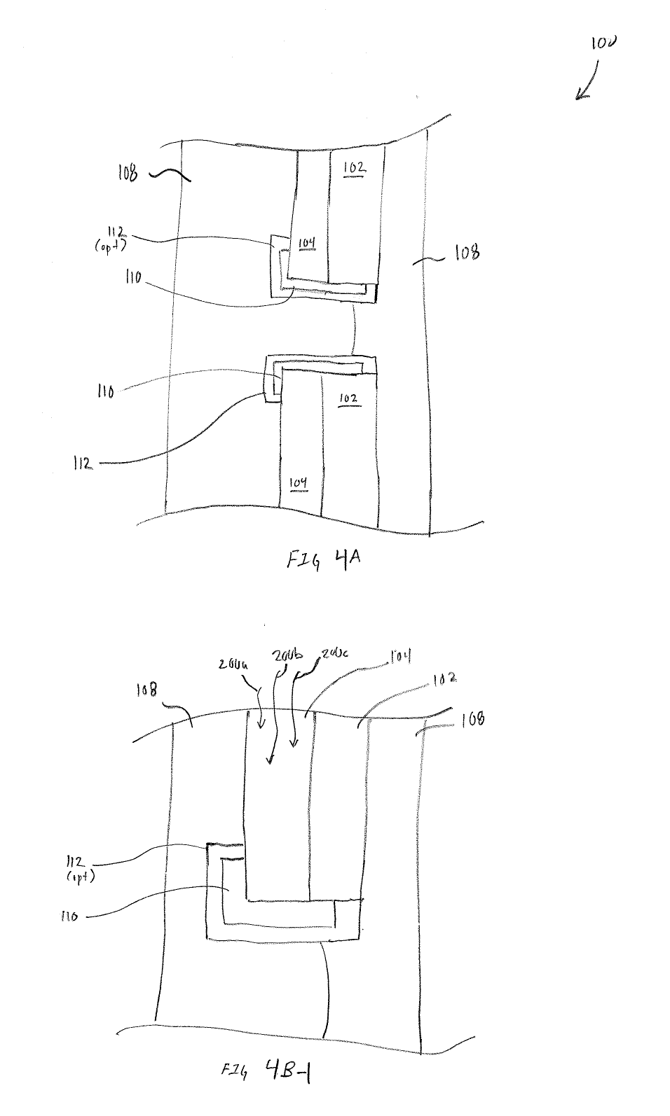

FIG. 5 is an exemplary flowchart illustrating operation to create a sensor assembly similar to what is illustrated in FIG. 3B-5, in accordance with embodiments of the present invention. The operations and order of operations discussed below should not be construed as limiting. The different embodiments illustrated in the Figures can each require execution of operations in varying orders or even additional operations such as, but not limited to masking, demasking and the like. The flowchart begins with operation 500. Operation 502 exposes a portion of the working conductor. Operation 504 applies the reactive surface to the working conductor. In many embodiments the application of the reactive surface involves multiple operations such as, but not limited to, electroplating and screen printing. However, in some embodiments, operation 504 is optional because the exposed working conductor is sufficient.

Operation 506 applies the second reactive chemistry over the working conductor and optional reactive surface. As previously discussed the second reactive chemistry can be mixed with a hydrogel. Additional materials can be mixed with the hydrogel to control porosity and thickness. In some embodiments, operation 506 further includes drying the second reactive chemistry but refrains from fully crosslinking the hydrogel. In a ketone sensor embodiment, operation 506 applies a mixture of NADH-oxidase and hydrogel over the working conductor.

Operation 508 applies the first reactive chemistry that includes a hydrogel to encapsulate the second reactive chemistry. in many embodiments, the first reactive chemistry further includes optional cofactor. Similar to the application of the second reactive chemistry, the first reactive chemistry is allowed to dry resulting in the first reactive chemistry not being fully crosslinked. In a ketone sensor embodiments, operation 508 encapsulates the second reactive chemistry under a mixture of NADH, NAD⁺, and hydrogel.

Operation 510 blanket coats the previously applied layers under the first transport material. The first transport material may be fully crosslinked. The cure cycle that fully crosslinks the first transport material may enable additional crosslinking of the previously applied layers thereby creating a gel like structure capable of swelling when hydrated when inserted into a subject. In a ketone sensor embodiments, operation 510 applies a hydrogel layer that is fully cured and crosslinked over the previously applied layers. The curing of the first transport material may create a gel like material by partially crosslinking the materials applied in operation 506 and 508.

Operation 512 applies the second transport material over the previously applied materials. In many embodiments, operation 512 applies a hydrophobic material such as, but not limited to, silicone thereby creating a no flux boundary between the first transport material and the second transport material. In embodiments measuring ketones, operation 512 applies a blanket layer of silicone over the previously applied materials. The specific operations described should not be construed as limiting or inclusive. Other embodiments may require more or fewer operations to create a sensor assembly. Furthermore, the application of materials described in the previously discussed operations should be construed broadly to encompass a variety of techniques, such as, but not limited to ink jet printing, deposition, screen printing, and the like.

The previously discussed operations are intended to be exemplary non-limiting operations intended to create a structure illustrated in FIG. 3B-5. The operations necessary to create the various layers illustrated in other Figures may require identical, similar or different operations. For example, while the operations described above were mostly additive, other embodiments may require operations that remove materials and/or require masking and demasking to enable placement of various layers/chemistries in particular locations.

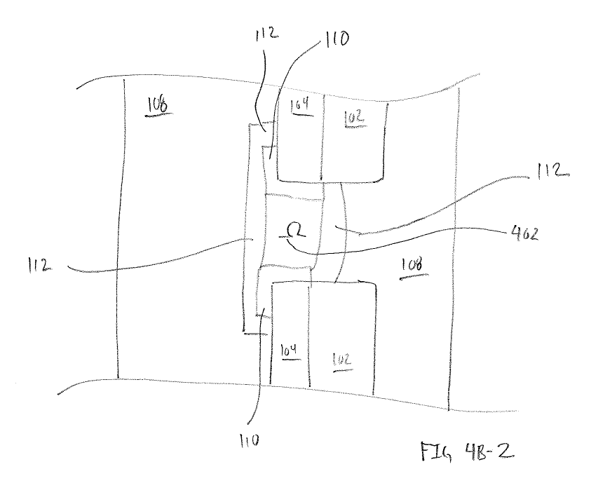

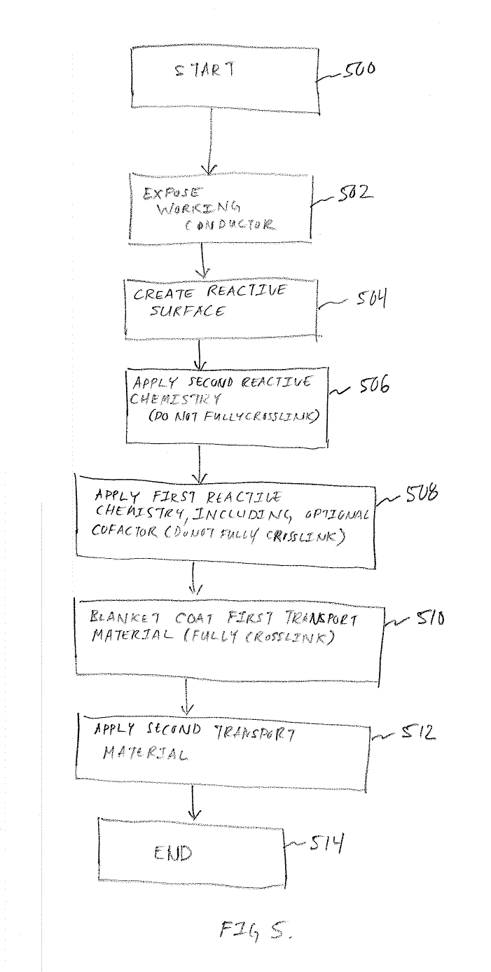

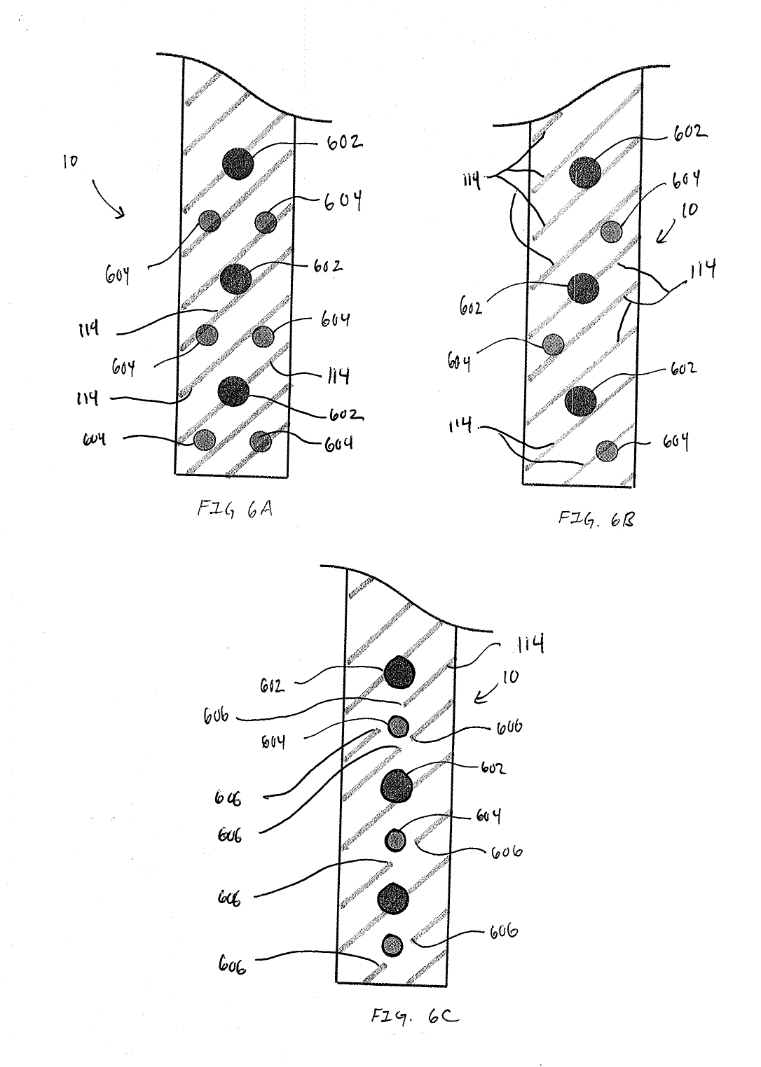

FIGS. 6A-6E are exemplary illustration of multianalyte sensor assemblies, in accordance with embodiments of the present invention. The discussion above is generally related to transducers that utilize a dehydrogenase component within the first reactive chemistry. As discussed, an exemplary non-limiting example would be a ketone sensor to detect concentrations of 3HB utilizing 3HBDH. The ability to simultaneously measure multiple analytes using a single sensor assembly can enable greater insight into the microcirculation of a subject. In many embodiments, the transducers described above are intended to be integrated with at least one other transducer configured to measure a different analyte such as, but not limited to glucose, lactate and/or oxygen. The inclusion of additional transducers, especially transducers utilizing reactive chemistries other than dehydrogenases, can impact design and layout of the sensor assembly. For example, in sensor assemblies measuring ketones via dehydrogenase and glucose via glucose oxidase, it may be advantageous to vary elements such as, but not limited to transducer size, transducer location (relative to transducer measuring a different analyte), placement of sensor elements (e.g., cofactor enhancing elements, second reactive chemistry, second transport material, and the like).

FIG. 6A and FIG. 6B are exemplary embodiments illustrating non-limiting top views of transducer placement on a sensor assembly 10 that is configured to measure at least two different analytes, in accordance with embodiments of the present invention. A first analyte is intended to be measured or detected using transducers 602. In some embodiments, transducers 602 utilize a reactive chemistry based on oxidase materials. A second analyte is intended to be measured or detected using transducers 604. In some embodiments, transducers 604 utilize a reactive chemistry based on dehydrogenase materials. Second transport material 114, illustrated as diagonal cross-hatching, blanket coats the entirety of the top surface of the sensor assembly 10.

In FIGS. 6A and 6B, placement of the transducers 604 closer to the edges 14a/14b of the sensor assembly 10 can be beneficial if the concentration of the analyte being detected by transducer 604 is low relative to the concentration of analyte being detected by transducer 602. Locating the transduces 604 toward the edges 14a/14b can improve response time and sensitivity of the transducer as well. The embodiment shown in FIG. 6B, where the dehydrogenase based transducers 604 are skewed toward one of the edges 14a/1b can enable faster response times. In FIG. 6C, breaks in cross-hatching 606 in close proximity to transducers 604 are intended to represent opening within the second transport material 114. In FIG. 6C, rather than blanket coating the entirety of the top of the sensor assembly 10, areas over the transducers 604 remain exposed. Accordingly, the embodiment in FIG. 6C accomplishes faster analyte access to the transducer 604 not by relative position toward the edges 14a/14b, but my removing the second transport material 114.

FIGS. 6D and 6E are exemplary, non-limiting embodiments of a multianalyte sensor assembly 10 that further includes at least one of IRM 304 or cofactor enhancing features 608a, 608b, and 608c, in accordance with embodiments of the present invention. In FIG. 6D transducer 602 measures a first analyte and transducers 604 measure a second analyte. Second transport material 114 blanket coats the top surface of the sensor assembly 10 creating a transport channel through the edges 14a/14b. Because all analyte enter the sensor via the exposed edges, the IRM 304 or cofactor enhancing feature 608a is located between the edges and the transducers 602 and 604. It may be desirable to utilize IRM 304 to mitigate migration of interfering compounds. Alternatively, it may be desirable to utilize a cofactor enhancing feature to increase efficiency, signal response, linearity or other aspects of transducer performance.

In FIG. 6E the cofactor enhancing feature 608b/608c or IRM 304 is placed closer to transducer 602/604. An alternative view would be that the transducers are placed in closer proximity, in this particular embodiment, concentric with the cofactor enhancing feature 608b/608c or IRM 304. As illustrated in FIG. 6E, with the transducer 604, being hydrogenase based, cofactor enhancing feature 608c can be used to generate cofactor. Alternatively, because of the proximity to transducer 602, an oxidase based sensor, rather than being a cofactor enhancing feature, IRM 304 including catalase is applied concentric with transducer 604. As previously discussed, in many embodiments, a cofactor enhancing feature may be an independently operated electrode that oxidizes particular analytes. In many embodiments, electrochemical cofactor enhancing features can used in conjunction with IRM 304 and chemistry based cofactor enhancing features. The particular embodiments illustrated in FIGS. 6A-6E are not intended to be inclusive. Previously discussed embodiments or combinations of embodiments including IRM and cofactor enhancing features can be optimized for use detecting a single analyte or multiple analytes.

While the description above refers to particular embodiments of the present invention, it will be understood that many modifications may be made without departing from the spirit thereof. Additionally, while particular embodiments described above may have specific features, what is disclosed in one embodiments is intended to be able to be combined or mixed with the other embodiments. Furthermore, it is intended that the various embodiments and features disclosed above can be combined or mixed with other embodiments such as those disclosed in U.S. patent application Ser. No. 15/472,194, filed Mar. 28, 2017 and International Application Number PCT/US18/38984, filed on Jun. 22, 2018 to create a vast variety of robust sensor assemblies ranging from single analyte with different types or working electrodes to multiple analyte with like or dissimilar types of working electrodes. The particular examples provided are intended to be illustrative embodiments of the multitude of combinations possible. The specific theories of operation provided throughout the disclosure should not be considered limiting. Rather, the disclosure is being made without being bound by any particular theory of operation. Accordingly, the disclosed embodiments and associated theories of operation are intended to be considered in all respects as illustrative and not restrictive.

Accordingly, while the description above refers to particular embodiments of the invention, it will be understood that many modifications may be made without departing from the spirit thereof. The presently disclosed embodiments are therefore to be considered in all respects as illustrative and not restrictive, the scope of the invention being indicated by the appended claims, rather than the foregoing description, and all changes that come within the meaning and range of equivalency of the claims are therefore intended to be embraced therein.

1. An electrode measuring the presence of an analyte, comprising:
a working conductor having an electrode reactive surface;
a first reactive chemistry being responsive to a first analyte;
a first transport material that enables flux of the first analyte to the first reactive chemistry;
a separation chemistry between the first reactive chemistry and the first transport material, the separation chemistry minimizing mixing of the first reactive chemistry and the first transport material.

2. The electrode described in claim 1, wherein the first reactive chemistry does not include a cofactor.

3. The electrode described in claim 1, wherein the first reactive chemistry includes a cofactor, the cofactor being responsive to a second analyte.

4. The electrode described in claim 3, further including:
a cofactor enhancing feature.

5. The electrode described in claim 4, wherein the cofactor enhancing feature is an amplifying electrode, the amplifying electrode generating the cofactor via oxidation of an endogenous analyte.

6. The electrode described in claim 4, wherein the cofactor enhancing feature includes:
addition of a second reactive chemistry within the electrode, the second reactive chemistry generating the cofactor via a reaction with an endogenous analyte.

7. The electrode described in claim 6, wherein the second reactive chemistry is selectively applied at least at a single discrete location within the electrode.

8. The electrode described in claim 6, wherein the second reactive chemistry is distributed through at least one of the first transport material or the second transport material.

9. The electrode described in claim 1, wherein the separation chemistry further enables selective transport of analyte between the first reactive chemistry and the first transport material.

10. The electrode described in claim 1, further including an interference reduction material.

11. The electrode described in claim 10, wherein the interference reduction material is selected based on an ability to reduce an endogenous analyte.

12. The electrode described in claim 4, further including an interference reduction material.

13. The electrode described in claim 12, wherein the interference reduction material is selected based on ability to reduce an analyte generated by a reaction between an endogenous analyte and the cofactor enhancing feature.

14. A method to manufacture an electrode comprising:
patterning a conductor material to generate a working conductor;
creating a reactive surface on the working conductor;
applying an interference reduction material over the reactive surface;
applying a first reactive chemistry over the interference reduction material;
applying a first transport material over the first reactive chemistry; and
applying a second transport material over the first transport material.

15. The method to manufacture an electrode described in claim 14, wherein the reactive surface is a multilayer structure.

16. The method to manufacture an electrode described in claim 14, wherein the interference reduction material is selected to reduce an analyte created between a reaction between an endogenous analyte and the first reactive chemistry.

17. The method to manufacture an electrode described in claim 14 wherein the first reactive chemistry is selected from a family of dehydrogenase chemistries.

18. The method to manufacture an electrode described in claim 14, wherein the first transport material is hydrophilic.

19. The method to manufacture an electrode described in claim 18, wherein the second transport material is hydrophobic.

20. The method to manufacture an electrode described in claim 19, wherein the second transport material confines the transport pathway for analyte within the first transport material.
